(19) United States
(12) Patent Application Publication
(10) Pub. No.: US 2023/0290466 A1
(43) Pub. Date: Sep. 14, 2023

(54) CLOUD-BASED INFUSION MEDICATION FULFILLMENT

(71) Applicant: Inovalon, Inc., Bowie, MD (US)

(72) Inventors: Zach Williams, Bowie, MD (US); Tim Simmons, Bowie, MD (US); Mark Sasala, Bowie, MD (US); Ashley Diermeir, Bowie, MD (US)

(21) Appl. No.: 18/118,540

(22) Filed: Mar. 7, 2023

Related U.S. Application Data
(60) Provisional application No. 63/318,750, filed on Mar. 10, 2022.
(60) Provisional application No. 63/318,761, filed on Mar. 10, 2022.

(51) Int. Cl. G16H 20/10; G06Q 10/087; H04L 67/10

(52) U.S. Cl. CPC G16H 20/10; G06Q 10/087; H04L 67/10

(57) ABSTRACT

Methods, systems, and apparatus, including medium-encoded computer program products for cloud-based infusion medication fulfillment. A cloud-based medication fulfillment system can receive a request to fulfill an order for a medication of a type. When the customer is authorized to process requests via an enhanced medication workflow that includes legacy medication fulfillment steps for fulfilling medications that are not of the type as well as enhanced medication fulfillment steps for fulfilling medications that are of the type, the system can: (i) process the request according to the legacy medication fulfillment steps, and (ii) process the request according to the enhanced medication fulfillment steps, including processing a representation of the request by an automated device that is associated with preparing medications of the type. The cloud-based medication fulfillment system can store data indicating that the order for the medication of the type has been fulfilled and/or administered.

### CROSS-REFERENCE TO RELATED APPLICATION

This application claims the benefit of U.S. Pat. App. No. 63/318,750, filed Mar. 10, 2022, and U.S. Pat. App. No. 63/318,761, filed Mar. 10, 2022, which are incorporated herein by reference.

### TECHNICAL FIELD

This specification relates to a cloud-based infusion medication fulfillment, including, in some implementations, techniques for controlling an automated compounding device using real-time, standardized data.

### BACKGROUND

In infusion therapy, medications are administered intravenously by injecting a needle directly into a patient's arm. Infusion therapy enables more efficient patient treatment, including the treatment of chronic illnesses, by delivering medicine, antibiotics and/or hydration directly into the bloodstream. In some cases, the infused medications are created using compounding, which can include creating a drug mixture to fit the unique needs of a patient.

### SUMMARY

This specification describes technologies relating to fulfilling infusion medication prescriptions. The techniques can include legacy approaches to fulfilling infusion medications and enhanced fulfillment techniques.

Pharmacies can operate according to structured workflows that are designed to ensure that the medication delivered to a patient is safe and effective. Such workflows can be managed by an automated orchestration process that guides the delivery of prescriptions from order receipt to medication fulfillment to billing. Such workflows are typically straightforward with one workflow operation following the prior operation, typically without considering real-time clinical data.

Unlike the operation of typical pharmacies, including specialty pharmacies, when preparing infusion medications, enhanced workflows, which can include significant complexity and can leverage real-time clinical data, are beneficial to ensure that strict rules that protect the health and welfare of a patient are consistently applied and enforced. Therefore, in some cases, based on the nature of the therapy and the benefit under which the therapy is covered, the fulfillment of a prescription may follow a legacy specialty pharmacy workflow in which the drug is known up-front, pharmacy coverage is established, the dispense is scheduled, claims are submitted as the order is processed, order completion is recognized, and the order is completed at shipping. However, because specialty pharmacies can also service infusion patients, and infusion pharmacies will dispense specialty medications created by following enhanced workflows, traditional and infusion workflows will overlap, especially in-light of the drug discovery pipelines, which increasingly includes infused therapies. For such reasons, there is a need for techniques that enable orders to be processed using hybrid fulfillment workflows that can include legacy and enhanced workflows.

In addition, as mentioned above, typical specialty pharmacies do not obtain and process real-time signals relating to patients and patient care, and therefore cannot adjust the preparation of medications in response to such signals. Even if such data were available, such records are often stored in a non-standard format selected by whichever hardware or software platform is in use in the medical provider's office. Therefore, a typical specialty pharmacy cannot obtain and utilize updated information about a patient's condition from other health care providers using current patient management systems. Further, since useful real-time data are unavailable, typical specialty pharmacies are limited in the verification of prescribed medication, considering only historical information such as medical history and previously-prescribed medications.

Particular embodiments of the subject matter described in this specification can be implemented so as to realize one or more of the following advantages. The techniques described below can be used to provide a cloud-based medication fulfillment that includes both legacy and enhanced fulfillment operations. By integrating legacy and enhanced operations, the system enables specialty pharmacies to provide infusion solutions, which have become increasingly important for patient care. In addition, the techniques can include using an automated compounding device to fulfill infusion medication requests, reducing the likelihood of human error, and therefore improving patient outcomes. The techniques can further include canonicalizing medical data, enabling the use of data from a broad range of external systems, even when such records are stored in non-standard formats used by the medical providers' offices. The techniques can also obtain and process real-time clinical data, which can be used to adjust the composition and/or delivery of infused medications.

One aspect features receiving, by a cloud-based medication fulfillment system, a request from a customer to fulfill an order for a medication of a particular type. When the customer is authorized to process requests via an enhanced medication workflow that includes one or more legacy medication fulfillment steps for fulfilling medications that are not of the particular type as well as one or more enhanced medication fulfillment steps for fulfilling medications that are of the particular type: (i) processing, by the cloud-based medication fulfillment system, the request according to the one or more legacy medication fulfillment steps, and (ii) processing, by the cloud-based medication fulfillment system, the request according to the one or more enhanced medication fulfillment steps, including processing a representation of the request by an automated device that is associated with preparing medications of the particular type. The cloud-based medication fulfillment system can store data indicating that the order for the medication of the particular type has been fulfilled.

One or more of the following features can be included. The automated device can be an automated compounding device. Processing the request according to the one or more enhanced medication fulfillment steps can include: (i) obtaining, by the cloud-based medication fulfillment system, within a threshold period of time after receiving the request, clinical data; (ii) performing, by the cloud-based medication fulfillment system, and using the clinical data, a verification; and (iii) in response to the verification, providing, by the cloud-based medication fulfillment system, a representation of the request to the automated device. The clinical data can be obtained in real-time. The verification can include a Drug Utilization Review (DUR). The verification can include processing an input that includes the clinical data by a machine learning model that is configured to verify the request. The cloud-based medication fulfillment system can adjust the ordered medication based on performing the DUR. Processing the representation can include sending the representation from the cloud-based medication fulfillment system to the automated device via an API that is established by the by the cloud-based medication fulfillment system. Processing the one or more enhanced medication fulfillment steps, by the cloud-based medication fulfillment system, can include, after preparing the medication by the automated compounding device and before the order is completed, decrementing an inventory of ingredients of the medication and incrementing an inventory of the medication. Processing the request according to one or more legacy medication fulfillment requests can include providing, to a third-party authorization system that is not included in the cloud-based medication fulfillment system, an indication that the order for the medication of the particular type has been fulfilled. Processing the request according to one or more legacy medication fulfillment requests can include providing, to a third-party infusion-delivery scheduling system that is not included in the cloud-based medication fulfillment system, an indication that the order for the medication of the particular type has been fulfilled. The cloud-based medication fulfillment system can obtain, from a medication-administration device and though an Application Programming Interface provided by the cloud-based medication fulfillment system, an indication that the medication has been administered, and provide to an accounts-receivable system, a charge associated with preparing, and in some cases administering, the medication. The clinical data can be canonicalized. The cloud-based medication fulfillment system can generate a code that is associated with preparing medications of the particular type, and provide the code to the automated device.

The details of one or more embodiments of the subject matter described in this specification are set forth in the accompanying drawings and the description below. Other features, aspects, and advantages of the invention will become apparent from the description, the drawings, and the claims.

### BRIEF DESCRIPTION OF THE DRAWINGS

FIG. 1A shows an example of an environment for a cloud-based medication fulfillment system.

FIG. 1B shows an example enhanced fulfillment engine.

FIG. 2 is a flow diagram of an example process for cloud-based medication fulfillment.

Like reference numbers and designations in the various drawings indicate like elements.

### DETAILED DESCRIPTION

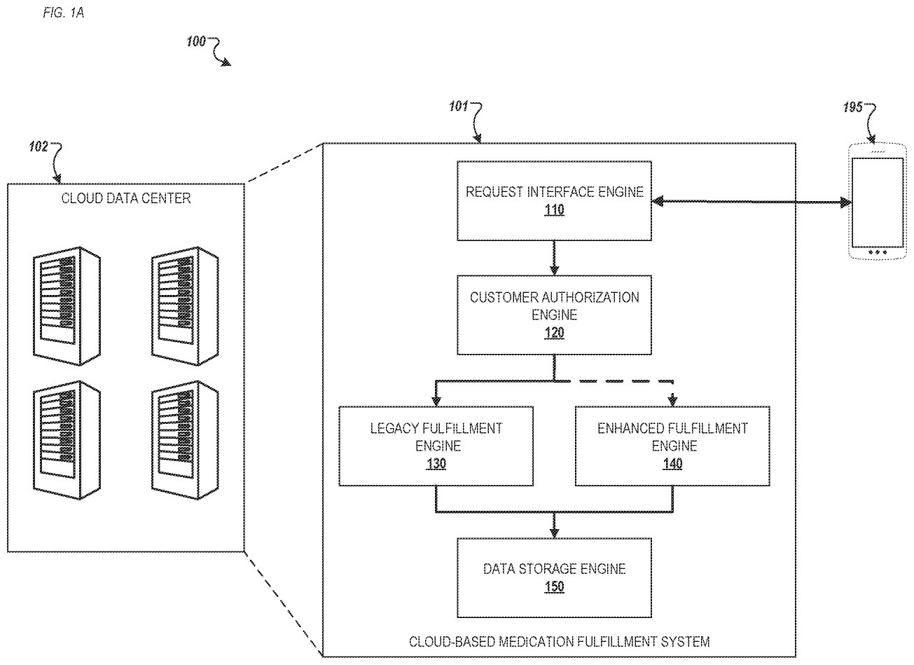

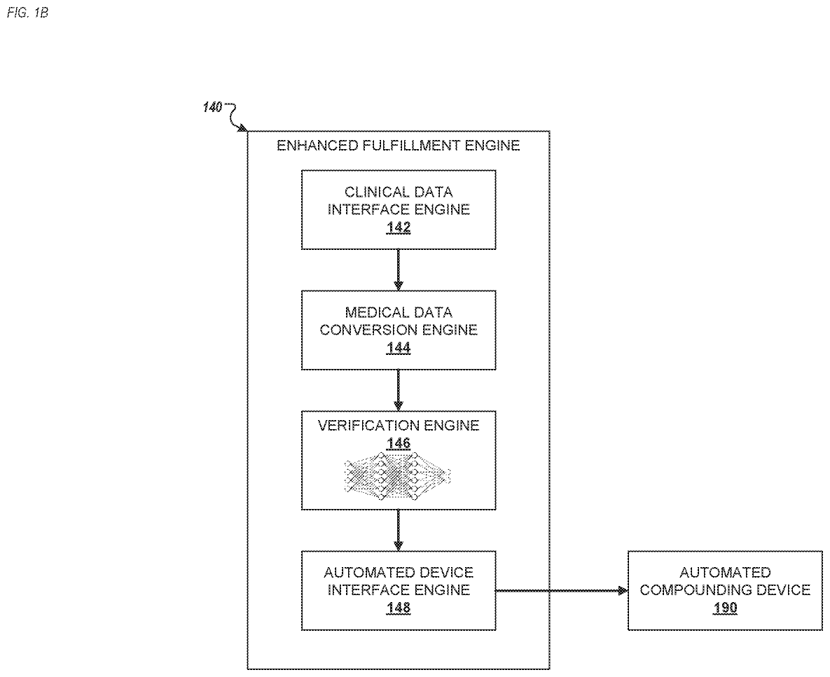

FIG. 1A shows an example of an environment for cloud-based medication fulfillment. The environment can support hybrid workflows that enable the fulfillment of both traditional medications and infusion medications, which are fluids, including medications, to be delivered directly into a patient's bloodstream.

Infusion pharmacies have many operational similarities with specialty pharmacies but benefit from unique functions and workflows. These intricate workflows, and the challenges of orchestrating such workflows, are primarily driven by the complexities of shorter, weekly dispense cycles, therapy adjustments based on weekly lab results, and the need to support medical benefits which often cannot be billed until after confirmed administration of treatment. Treatment can either be self-administered in-home, nurse-administered, or on-site in an infusion suite.

Additionally, there is a need to support referrals coming from discharge coordinators who often do not have a prescribed drug yet need to anticipate the planning of the patient's transition of care from in-patient to ambulatory or home infusion. The techniques described below support these objectives, including integrating legacy workflows for fulfilling traditional prescriptions and enhanced workflows for fulfilling infusion prescriptions to create hybrid workflows that can be orchestrated using a cloud-based system. The techniques further include the integration of real-time clinical data, which can improve patient outcomes, but can require complex data transformations to canonicalize disparate data formats.

Returning to FIG. 1A, the environment for cloud-based medication fulfillment 100 can include a cloud-based medication fulfillment system 101 that can be run in a cloud data center 102 and accept requests from client devices 195 associated with customers using the environment 100.

The cloud data center 102 can be a distributed computing system that includes computing resources such as servers, storage, networking, special-purpose processors (e.g., graphics processing units and field programmable gate arrays, among other examples) and other computing resources. The cloud data center 102 can exist in a single location or span multiple locations, which can be distributed worldwide. The cloud data center 102 can include hundreds or thousands of computers and can be configured to provide a highly-available computing platform on which the cloud-based medication fulfillment system 101 operates. In addition, the cloud data center 102 can provide elastic resource scaling such that the computing resources available to the cloud-based medication fulfillment system 101 are scaled to meet the varying demands of the cloud-based medication fulfillment system 101.

The cloud-based medication fulfillment system 101 can provide hybrid fulfillment workflows that include both legacy fulfillment workflows and enhanced fulfillment workflows. To provide such hybrid workflows, the cloud-based medication fulfillment system 101 can include a legacy fulfillment engine 130 and an enhanced fulfillment engine 140. The cloud-based medication fulfillment system 101 can further include a request interface engine 110, a customer authorization engine 120, and a data storage engine 150. The cloud-based medication fulfillment system 101 can also include, or be coupled to, an automated device for fulfilling prescriptions according to a recipe, such as an automated compounding device, as described further in reference to FIG. 1B. In some implementations, the cloud-based medication fulfillment system 101 can further include, or be coupled to, an adherence packaging system, which can to organize medication, such as pills, into dosing times and dates, helping patients take the right doses at the right times.

The request interface engine 110 can accept requests for medications from customers, who send the requests on behalf of patients. The customer can be the party that will administer the medication, the prescriber of the medication, another pharmacy, a medical facility, a discharge coordinator, among other examples.

A request can include a prescription and related documentation. The prescription can include the name of the medication, patient name or other patient identifier, amount of the medication, concentration of active ingredients, overfill amount, among other data. The related documentation can include a certificate of medical necessity, a durable medical equipment form, the date needed, the prescriber, location the medication will be dispensed,

A client device 195 is an electronic device that is capable of communicating over a network, e.g., with a cloud data center 102. Example client devices 195 include personal computers, server computers, mobile communication devices, e.g., smart phones and/or tablet computers, and other devices that can send and receive data over the network. A client device 195 can include applications, such as web browsers and/or native applications, to facilitate the sending and receiving of data over the network. A native application is an application developed for a particular platform or a particular device (e.g., mobile devices having a particular operating system). Although operations may be described as being performed by the client device 195, such operations may be performed by an application running on the client device 195.

The customer authorization engine 120 can determine whether the customer is authorized to use the enhanced fulfillment techniques. The customer authorization engine can include an access control list that includes a list of customers authorized to use the legacy techniques and customers authorized to use the enhanced techniques. When a customer is authorized to use the legacy techniques, the request can be passed to the legacy fulfillment engine 130. When a customer is authorized to use the enhanced techniques, the request can be passed to the enhanced fulfillment engine 140. If the customer is authorized to use both legacy and enhanced techniques, the request can be passed to both the legacy fulfillment engine 130 and the enhanced fulfillment engine 140. In some implementations, authorization to use the enhanced fulfillment techniques is sufficient to authorize the use of the legacy fulfillment techniques.

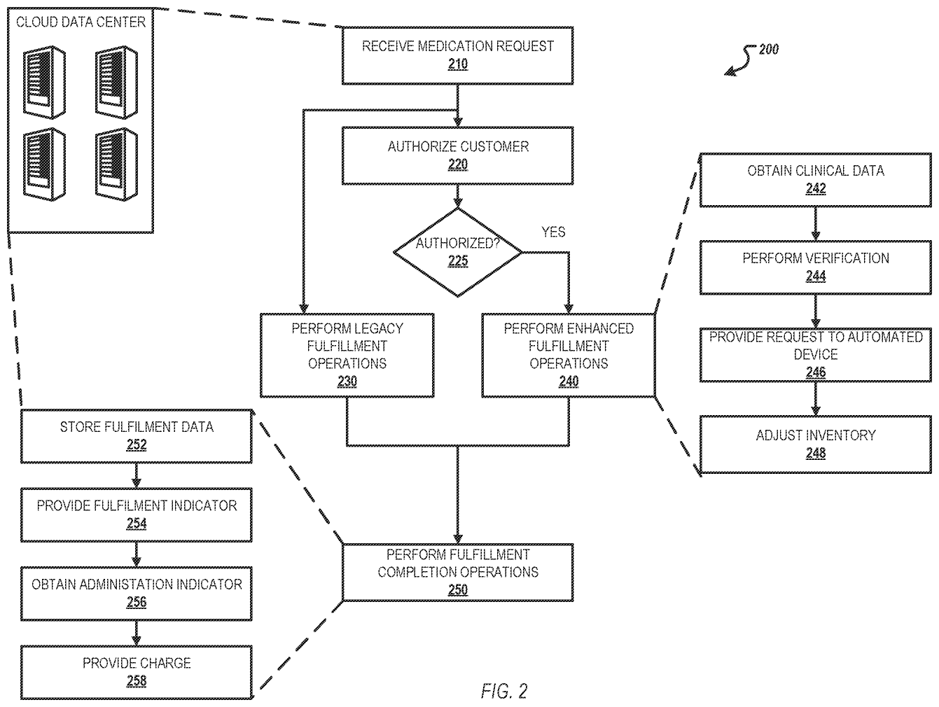

The legacy fulfillment engine 130 can obtain the request and perform the legacy fulfillment techniques as described further in reference to FIG. 2. Upon completion of a request, the legacy fulfillment engine 130 can provide information describing the fulfillment to the data storage engine 150.

FIG. 1B shows an example enhanced fulfillment engine 140. The enhanced fulfillment engine 140 can include a clinical data interface engine 142, a medical data conversion engine 144, a verification engine 146 and an automated device interface engine 148. In addition, the enhanced fulfillment engine 140 can include, or be coupled to, an automated compounding device 190.

The clinical data interface engine 142 can accept clinical data from entities that include data relating to the patient. Clinical data can include vital signs (blood pressure, pulse, temperature, etc.); height; weight, gender; disease history; current, prior and planned medications; treatment history; among other examples. The clinical data interface engine 142 can include an application programming interface (API) that enables authorized parties to provide the clinical data. The API can be, for example, a web services API or a remote procedure call API. Examples of authorized parties can include the provider of the medication, other medical providers for the patient, devices measuring patient information (e.g., heart-rate and blood pressure monitors), medical record archives, etc. Upon receipt of clinical data, the clinical data interface engine 142 can associate a time stamp with the clinical data, and the time stamp can be used to determine the age of the data when making determinations.

The clinical data can be historical data, real-time data or a combination of historical and real-time data. Real-time data can be defined as clinical data received within a threshold time period of receiving the request. For example, the threshold can be 10 seconds, 30 seconds, 1 minute, etc. Historical data can be any clinical data that is not received within the threshold period, including data that is stored by the system in a secure archive.

The medical data conversion engine 144 can convert the received clinical data into a canonical format. Since clinical data records can be provided in non-standard formats selected by whichever hardware or software platform is in use in medical providers' local office, which would create challenges for the enhanced fulfillment engine 140 to use the clinical data, the medical data conversion engine 144 converts the data to a usable, canonical form. The medical data medical data conversion engine 144 can include a set of rules that define how data from each non-standard format is mapped into the canonical format used by the enhanced fulfillment engine 140. The mapping techniques are described in reference to FIG. 2, e.g., in reference to operation 242.

The verification engine 146 can use the clinical data to determine whether the requested medication is safe and effective to administer. In some implementations, the system can include a set of rules that indicate whether the medication is predicted to be safe and effective. The rules can depend on the clinical data, the medication, historical data related to the patient and prior treatment, among other factors.

In some implementations, the system can include a machine learning model that is configured to predict whether the medication is predicted to be safe and effective. The machine learning model can be, for example, a deep neural network, a decision tree, a support vector machine or other appropriate type of machine learning model.

The automated device interface engine 148 can provide instructions for preparing the medication to an automated compounding device 190. The automated device interface engine 148 can transform a canonical representation of the medication used by the cloud-based medication fulfillment system 101 to a format used by the automated compounding device 190. Note that each automated compounding device 190 can use a different format, so the automated device interface engine 148 must be configured to transform the canonical format into multiple device-specific formats.

The automated compounding device 190 aseptically transfers precise amounts of one or more sterile component solutions to a sterile container where it can be used for patient care. Automated compounding devices 190 can decrease the need for measuring and delivering component solutions with syringed and reducing preparation time. Automated compounding devices 190 can provide multiple modes of operation, including gravimetric and the volumetric. The gravimetric automated compounding systems use specific gravity to determine the weight to be transferred. While a volumetric automated compounding system 190 determines the volume of solution that needs to be transferred to the final container. Automated compounding devices 190 can include multiple ports, which can be used to supply components of the compound being created.

Returning to FIG. 1A, in some implementations, the legacy fulfillment engine 130 and/or the enhanced fulfillment engine can convert from the prescribed medication to the “recipe” used to create the medication. The conversion can use a master formulation record, which can include, for example: official name, strength, and dosage; physical description of the final preparation; description of all ingredients and their quantities; complete instructions for preparing the CSP including equipment, supplies, and description of compound steps including sterilization method, if applicable; assigned Beyond-Use-Date (BUD) calculation compliant with USP standards (if extending BUD beyond the current USP standards, then references establishing compatibility and stability must be included); storage requirements; and quality control procedures (e.g., pH, sterilization method, and visual inspection).

The data storage engine 150 stores data describing the fulfillment, which can be called “fulfillment data,” on one or more data storage and/or data processing systems. The data describing the fulfillment can include information about the medication fulfilled, the ingredients used, amounts of each ingredient used, the source of the ingredients, the requesting customer, the patient, insurance information, cost, the time of fulfillment, the fulfilled medication expiration date, among many other examples.

The data storage engine 150 can store the fulfillment data on any appropriate storage system. For example, the data can be stored in a relational database or on a file system. In another example, the data can be provided to one or more systems configured to perform accounting, billing, schedule, reporting and/or other post-fulfillment tasks.

FIG. 2 is a flow diagram of an example process for cloud-based medication fulfillment. For convenience, the process 200 will be described as being performed by a system for cloud-based medication fulfillment, e.g., the cloud-based medication fulfillment system 101 of FIG. 1, appropriately programmed to perform the process. Operations of the process 200 can also be implemented as instructions stored on one or more computer readable media which may be non-transitory, and execution of the instructions by one or more data processing apparatus can cause the one or more data processing apparatus to perform the operations of the process 200. One or more other components described herein can perform the operations of the process 200.

The system can receive (210) a request from a customer to fulfill an order for a medication of a particular type. In some implementations, the system can include an API, which, when called by a customer, enables a customer to provide a request. The system can also receive messages from the customer that include the request.

The system can authorize (220) a customer for processing of requests via an enhanced medication workflow. The enhanced workflow can be component of a hybrid workflow—that is, the workflow can include a legacy workflow that includes one or more legacy medication fulfillment steps for fulfilling medications, e.g., medications that are not of the particular type (e.g., they are not infusion medications), as well as one or more enhanced workflows that include enhanced medication fulfillment steps for fulfilling medications that are of the particular type (e.g., infusion medications).

The system can maintain a list of customers that are authorized to the enhanced medication workflow, and the system can compare the customer identified in the request to the customers who are included in the list of authorized customers. If the customer included in the request is in the list of authorized customers, the system can determine that the customer is authorized, and otherwise determine that the customer is not authorized.

In some implementations, to provide additional security, the system can require verification of a customer's identity. For example, the system can prompt the customer for a password and/or use other authentication techniques.

Customers can be added to the authorization list using various techniques and criteria. In some implementations, customers can be authorized based on business relationships. For example, a customer might register to use the enhanced workflow, which can, in some cases, include an additional fee. In some implementations, the system can determine based on the request whether the enhanced workflow is required to handle the request. For example, if the request relates to an infusion medication, the customer can be added to the authorization list for the purpose of fulfilling the request. In some implementations, the system can attempt to process the request using the legacy workflow, and if the legacy workflow reports an error, the system can add the customer to the authorization list.

In some implementations, the list can further include indications of medications and/or prescribed administration method (e.g., how the medication is packaged and delivered). Therefore, the system can, in some implementations, determine not only whether the customer is generally authorized to use the enhanced medication workflow, but whether the customer is authorized to use the enhanced medication workflow for a particular medication and/or prescribed administration method.

If the customer is authorized (225), the system can perform the enhanced medication workflow of operation 240. Regardless of whether the customer is authorized, the system can perform the legacy medication workflow of operation 230.

The system can process (230) the request according to the one or more legacy medication fulfillment steps using the legacy workflow that is part of the hybrid fulfillment workflow. The legacy fulfillment steps can include intake coordination, preliminary quality assurance (QA) and pharmacist verification.

Intake coordination enables the pharmacy to track and work inbound referrals with no definitive prescribed drug, which is important since the pharmacy's benefits investigation can influence both the site of care as well as the selected drug. Intake coordination can include the evaluation of relevant data, which can include a Certificate of Medical Necessity (CMN) and/or a Durable Medical Equipment (DME) Information Form (DIF), which are forms required to help document the medical necessity and other coverage criteria for selected durable medical equipment, prosthetics, orthotics, and supplies (DMEPOS) items. The data can further include Advance Beneficiary Notice of Noncoverage (ABN), which is issued by providers (including independent laboratories, home health agencies, and hospices), physicians, practitioners, and suppliers to Original Medicare (fee for service—FFS) beneficiaries in situations where Medicare payment is expected to be denied.

As part of intake coordination, the system can provide to an authorized party, such as a pharmacy technician or care coordinator, information about the request. The authorized party can use the request to coordinate with hospital personnel (e.g., a discharge coordinator) to smooth the transition of care, ensure coverage is established and affordable, and confirm that the need date is appropriate for delivering the drug to the patient in time for their first home or ambulatory infusion once they are discharged. The system can accept from the authorized party performing intake coordination an indication that coordination is complete.

Preliminary QA can include a drug utilization review (DUR). A DUR determines whether prescriptions for drugs are appropriate, medically necessary, and not likely to result in adverse medical consequences. A DUR can be implemented as a set of rules, such as a subset of the rules used for verification, which indicate whether a particular medication satisfies the DUR. If a medication does not satisfy the DUR, the system can provide a notification (e.g., by secure message, email, text message, etc.) to the customer, the patient, the patient's caregivers, among other parties.

In some implementations, the legacy fulfillment steps include pharmacist verification. The system can provide a representation of the request to a system accessible by the pharmacist, and the system can receive from the pharmacist an indication as to whether the request is predicted to be safe and effective for the patient. In addition, the pharmacist can edit the medical orders as appropriate and authorized, e.g., by the prescriber-defined protocol. In some implementations, such adjustments can initiate additional workflows, such as requesting approval from the initial prescriber. Further, the pharmacist can enter additional data or additional Prescription (Rx) Lines (Manage Lines). For infusion therapies, pharmacists often have a higher level of clinical expertise than technicians, and pharmacist entry of numerous prescription (and service) data may be advantageous.

The legacy fulfillments steps can also optionally include operations such as order processing and other administrative tasks. For example, the legacy fulfillments steps can include providing data to billing systems.

The system can process (240) the request according to the one or more enhanced medication fulfillment steps using the enhanced workflow that is part of the hybrid fulfillment workflow. In some implementations, the enhanced fulfillment steps can include obtaining clinical data, performing verification, and providing a representation of the request to an automated device and/or adjusting inventory.

The system can obtain (242) clinical data, which can include data describing the patient. As described above, the system can obtain the clinical data by providing an API that enables authorized parties to provide the clinical data. The system can verify that the entity is authorized by requiring secure credentials, such as an encrypted token or password, to be included in the API call and comparing the credentials against a list of authorized credentials.

In some implementations, the system can accept clinical data in various data formats. For example, one medical provider might store clinical data in a first format, and a second medical provider might store clinical data in a second format. The system can include a canonical format for clinical data, e.g., expressed as in Extensible Markup Language (XML), and transformation rules that map the syntax of non-canonical formats to the canonical format. For example, a non-standard data format might specify patient weight using the tags <WEIGHT></WEIGHT> whereas the canonical format might use <PATIENT_WEIGHT></PATIENT_WEIGHT>. The system can include a rule that indicates when the token “WEIGHT” is encountered, the system will replace that token with “PATIENT_WEIGHT.”

Other rules that perform structural transformations can also be used. For example, if the non-standard data format includes outer tag <PATIENT></PATIENT> and inner tags such as <WEIGHT></WEIGHT> and <HEIGHT></HEIGHT>, a rule can specify the mapping as show in Listing 1, below

LISTING 1

```
Non-canonical form
<PATIENT>
  <WEIGHT> 150 </WEIGHT>
  <HEIGHT> 66 </HEIGHT>
</PATIENT>
Canonical form
<PATIENT_WEIGHT> 150 </PATIENT_WEIGHT>
<PATIENT_HEIGHT> 66 </PATIENT_HEIGHT>
```

In another example, semantic elements of a schema might differ from the semantic elements of the canonical schema. In response, as part of the conversion to the standard format, the system can perform semantic conversion. In some implementations, the system can include rules that map semantics of non-canonical data formats to semantics of the canonical data format. For example, the system can canonicalize data format to use metric measurements such that if a provider supplies clinical data regarding weight in pounds, and the canonical format defines weight in kilograms, the transformation can specify that the weight in pounds is to be divided by 2.205 to obtain the weight in kilograms.

In some examples, the system will perform both syntactic and semantic transformation. Using the examples given above, if the non-canonical for uses tags, <WEIGHT></WEIGHT>, and those weights are given in pounds, the system would both convert the tags to <PATIENT_WEIGHT></PATIENT_WEIGHT> and divide the value representing the weight by 2.205.

Further, the syntactic transformation is not limited to XML-to-XML mapping. For example, the non-canonical format might be expressed in JavaScript Object Notation (JSON), other text-based formats, binary formats, and so on. The system can include parsing rules for each known incoming format, and rules that transform such formats to the canonical format.

Since different data sources can use different data encodings, each of which might or might not be the canonical format, the system can identify the data source, and use the data source to identify the appropriate mapping from non-standard to canonical format. In some implementations, the system can identify the sender (e.g., using a field in the communication that identifies the sender), and use the identity of the sender to determine the data transformation rules. In some implementations, if the system does not include appropriate transformation rules, the system can use the identity of the sender to obtain (e.g., by downloading from a web site or database) transformation rules. Note that the system need only download rules that describe the transformation from the format used by the sender to another known format, not necessarily to a transform that maps directly to the canonical format. For example, if the canonical format is F_{c} and the source format is Fₛ, then the system can download a transform from Fₛ to F_{c}. If such a direct transform is not available, and the system includes a transform from Fₛ′ to F_{c}, then the system can download a transform from Fₛ to Fₛ′, then perform the mapping Fₛ->Fₛ′->F_{c}. Any number of intermediate transformations can be used.

The system can perform (244) verification using the clinical data. The system can evaluate a set of rules that define whether the requested medication is safe and effective to administer in light of the clinical data. (As described above, verification using the legacy techniques does not include evaluation of real-time clinical data as real-time clinical data is not available to the legacy operations.) If the evaluation of a rule indicates a safety concern the system can provide a notification. In some implementations, the system can cease processing if a safety concern is encountered. In some implementations, the system can provide a notification to an authorized party (e.g., a pharmacist), and proceed only if that party indicates that it is safe to proceed. In some implementations, the system can add an entry to an exception queue within the workflow, and the entry must be reviewed and relieved by an authorize party before the workflow can continue.

In some implementations, rules can include an indication of severity, and the severity can be used to determine actions. For example, if a severity indicates a high risk to health, the system can cease processing and provide an error notification. Lower levels of severity can result in the system providing informational notifications.

In some implementations, the verification can include an enhanced DUR that includes the clinical data. As described above, the DUR can be implemented as a set of rules, such as a subset of the rules used for verification, which indicate whether a particular medication satisfies the DUR in light of the clinical data. If a medication does not satisfy the DUR, the system can provide a notification (e.g., by secure message, email, text message, etc.) to the customer, the patient, the patient's caregivers, among other parties.

In some implementations, the rules can further specify adjustments to be made to the ordered medication, for example, as a result of the DUR. For example, the request can specify a medication be delivered at a first rate, and the rule can indicate that the medication is delivered at a specific rate that differs from the first rate. In another example, the delivery can be changed from gravity delivery to a pumped delivery at a specific rate.

In some implementations, verification can include using a machine learning model, such as a deep neural network. The system can process an input that includes the clinical data, the request, and other relevant content using a machine learning model that is configured to produce a verification result. The verification result can include an indication that the treatment is predicted to be safe or unsafe, an indication that the treatment is predicted to be effective, recommended modifications to the treatment to increase the predicted likelihood of safety and/or efficacy, among other possible predictions.

In response to the verification, the system can provide (246) a representation of the request to an automated device that is configured to prepare medications of the type indicated in the request. For example, the automated device can be an automated compounding device that transfers proper amounts of one or more components of the medication to a final container.

In some implementations, the system can map the medication indicated in the request to the recipe needed to formulate the recipe, and the system can provide the recipe to the automated device. In some implementations, the automated device is configured with appropriate recipes, and the system need only provide the medication name. If the automated device is not configured with the appropriate recipe, the system can provide the recipe instead of, or along with, the medication name.

In some implementations, the system can provide the representation to the automated device by calling an API provided by the device. In some implementations, the system can provide an API, which, when called by the automated device, provides the representation from the system to the automated device. In some implementations, the automated device can include an API, which, when called by the system, informs the automated device that a representation is available, and the automated device can call an API provided by the system to retrieve the representation, e.g., when the automated device is available.

In some implementations, the system can generate a code that represents the medication and/or representation and provide the code to the automated device. The code can be, for example, a bar code, a quick response (QR) code or an audio tone. In various implementations, the system can print a code and provide the printed code at a location accessible to the automated device; display a code on a display device (e.g., monitor) that is accessible to the automated device; generate an audio tone within reception range of the automated device; and so on.

In some cases, the form of the request can differ from the data representation required by the automated device. In such cases, the system can transform the data from the format used in the request to the format required by the automated device. The system can use the techniques described in reference to operation 242, or similar data transformation techniques. Thus, the system can include multiple types of data transformation, such as the transformation of clinical data into a canonical format, and the transformation of data to automated-device-specific formats.

The system can adjust (248) the inventory of the medication and/or the ingredients. After preparing the medication, e.g., using an automated device such as an automated compounding device, and before the order is completed, the system can decrement an inventory of the ingredients used to prepare the medication and/or increment an inventory of the compound medication. In some implementations, the ingredients used to prepare the medication, and the amounts of each medication used, can be determined from the recipe for the drug included in the request. In some implementations, the automated device can provide the actual amounts of each ingredient used. In some implementations, the system can include an API available to an authorized user (e.g., a pharmacist) who provides a list of ingredients used and amounts of each ingredient used.

The system can perform (250) fulfillment completion operations, which can, in various implementations, include storing fulfillment data, providing one or more fulfillment indicators, obtaining an administration indicator and/or providing charging information, as described further below.

The system can store (252) data indicating that the order for the medication of the particular type has been fulfilled. The system can store the data in any appropriate repository such as an order tracking system, a relational database, an object storage system, a block storage system and so on.

The system can provide (254) an indication that the order for the medication of the particular type has been fulfilled. The system can include a list of parties to which such indications are to be provided, and can provide the indications using various techniques. In some implementations, the list can include a preferred indication technique. For example, a party to receive the indication can provide a uniform resource locator (URL) of an API to which the indication is to be sent. In another example, the party can indicate an e-mail address or other address type to which the indication should be sent. The system can provide the indication using the preferred indication technique for each party in the list. In some implementations, the indication can be sent to a third-party authorization system that is not included in the cloud-based medication fulfillment system, such as an insurance company. In some implementations, the indication can be provided as part of the legacy fulfillment operations (e.g., as described in reference to operation 230) in addition to, or instead of, providing the indication as part of the enhanced fulfillment operations.

The system can obtain (256) from a medication-administration device and through an API provided by the system, an indication that the medication has been administered. The API can be, for example, a web services API or remote procedure call (RPC) API. In some implementations, the indication that the medication has been administered can be received from an authorized user of the system, e.g., through a graphical user interface provided by the system.

The system can provide (258) to an accounts-receivable system, which can be part of the system or external to the system, a charge associated with preparing the medication. The system can provide an indication of the charge to the account receivable system using various techniques. For example, the system can provide an API that, when called by the accounts-receivable system, provides the charge from the system to the accounts-receivable system. In another example, the system can call an API provided by the accounts-receivable system.

An example computer system that can be used to perform operations described above can include a processor, a memory, a storage device, and an input/output device. Each of the components can be interconnected, for example, using a system bus. The processor is capable of processing instructions for execution within the system. In one implementation, the processor is a single-threaded processor. In another implementation, the processor is a multi-threaded processor. The processor is capable of processing instructions stored in the memory or on the storage device.

The memory stores information within the system. In one implementation, the memory is a computer-readable medium. In one implementation, the memory is a volatile memory unit. In another implementation, the memory is a non-volatile memory unit.

The storage device is capable of providing mass storage for the system. In one implementation, the storage device is a computer-readable medium. In various different implementations, the storage device can include, for example, a hard disk device, an optical disk device, a storage device that is shared over a network by multiple computing devices (e.g., a cloud storage device), or some other large capacity storage device.

The input/output device provides input/output operations for the system. In one implementation, the input/output device can include one or more of a network interface devices, e.g., an Ethernet card, a serial communication device, e.g., and RS-232 port, and/or a wireless interface device, e.g., and 802.11 card. In another implementation, the input/output device can include driver devices configured to receive input data and send output data to other input/output devices, e.g., keyboard, printer and display devices. Other implementations, however, can also be used, such as mobile computing devices, mobile communication devices, set-top box television client devices, etc.

Although an example processing system has been described above, implementations of the subject matter and the functional operations described in this specification can be implemented in other types of digital electronic circuitry, or in computer software, firmware, or hardware, including the structures disclosed in this specification and their structural equivalents, or in combinations of one or more of them.

Embodiments of the subject matter and the functional operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented using one or more modules of computer program instructions encoded on a computer-readable medium for execution by, or to control the operation of, data processing apparatus. The computer-readable medium can be a manufactured product, such as a hard drive in a computer system or an optical disc sold through retail channels, or an embedded system. The computer-readable medium can be acquired separately and later encoded with the one or more modules of computer program instructions, such as by delivery of the one or more modules of computer program instructions over a wired or wireless network. The computer-readable medium can be a machine-readable storage device, a machine-readable storage substrate, a memory device, or a combination of one or more of them.

The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, a runtime environment, or a combination of one or more of them. In addition, the apparatus can employ various different computing model infrastructures, such as web services, distributed computing and grid computing infrastructures.

A computer program (also known as a program, software, software application, script, or code) can be written in any suitable form of programming language, including compiled or interpreted languages, declarative or procedural languages, and it can be deployed in any suitable form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub-programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, special purpose microprocessors. Generally, a processor will receive instructions and data from a read-only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM (Erasable Programmable Read-Only Memory), EEPROM (Electrically Erasable Programmable Read-Only Memory), and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

In this specification the term “engine” is used broadly to refer to a software-based system, subsystem, or process that is programmed to perform one or more specific functions. Generally, an engine will be implemented as one or more software modules or components, installed on one or more computers in one or more locations. In some cases, one or more computers will be dedicated to a particular engine; in other cases, multiple engines can be installed and running on the same computer or computers.

To provide for interaction with a user, embodiments of the subject matter described in this specification can be implemented on a computing device capable of providing information to a user. The information can be provided to a user in any form of sensory format, including visual, auditory, tactile or a combination thereof. The computing device can be coupled to a display device, e.g., an LCD (liquid crystal display) display device, an OLED (organic light emitting diode) display device, another monitor, a head mounted display device, and the like, for displaying information to the user. The computing device can be coupled to an input device. The input device can include a touch screen, keyboard and a pointing device, e.g., a mouse or a trackball, by which the user can provide input to the computing device. Other kinds of devices can be used to provide for interaction with a user as well; for example, feedback provided to the user can be any suitable form of sensory feedback, e.g., visual feedback, auditory feedback, or tactile feedback; and input from the user can be received in any suitable form, including acoustic, speech, or tactile input.

The computing system can include clients and servers. A client and server are generally remote from each other and typically interact through a communication network. The relationship of client and server arises by virtue of computer programs running on the respective computers and having a client-server relationship to each other. Embodiments of the subject matter described in this specification can be implemented in a computing system that includes a back-end component, e.g., as a data server, or that includes a middleware component, e.g., an application server, or that includes a front-end component, e.g., a client computer having a graphical user interface or a Web browser through which a user can interact with an implementation of the subject matter described is this specification, or any combination of one or more such back-end, middleware, or front-end components. The components of the system can be interconnected by any suitable form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

While this specification contains many implementation details, these should not be construed as limitations on the scope of what is being or may be claimed, but rather as descriptions of features specific to particular embodiments of the disclosed subject matter. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination. Thus, unless explicitly stated otherwise, or unless the knowledge of one of ordinary skill in the art clearly indicates otherwise, any of the features of the embodiments described above can be combined with any of the other features of the embodiments described above.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and/or parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

Thus, particular embodiments of the invention have been described. Other embodiments are within the scope of the following claims. For example, the actions recited in the claims can be performed in a different order and still achieve desirable results.

What is claimed is:

1. A computer-implemented method comprising:
receiving, by a cloud-based medication fulfillment system, a request from a customer to fulfill an order for a medication of a particular type;
when the customer is authorized to process requests via an enhanced medication workflow that includes one or more legacy medication fulfillment steps for fulfilling medications that are not of the particular type as well as one or more enhanced medication fulfillment steps for fulfilling medications that are of the particular type:
processing, by the cloud-based medication fulfillment system, the request according to the one or more legacy medication fulfillment steps, and
processing, by the cloud-based medication fulfillment system, the request according to the one or more enhanced medication fulfillment steps, including processing a representation of the request by an automated device that is associated with preparing medications of the particular type; and
storing, by the cloud-based medication fulfillment system, data indicating that the order for the medication of the particular type has been fulfilled.

2. The computer-implemented method of claim 1, wherein the automated device is an automated compounding device.

3. The computer-implemented method of claim 1, wherein processing the request according to the one or more enhanced medication fulfillment steps comprises:
obtaining, by the cloud-based medication fulfillment system, within a threshold period of time after receiving the request, clinical data;
performing, by the cloud-based medication fulfillment system, and using the clinical data, a verification; and
in response the verification, providing, by the cloud-based medication fulfillment system, a representation of the request to the automated device.

4. The computer-implemented method of claim 3, wherein the clinical data is obtained in real-time.

5. The computer-implemented method of claim 3, wherein the verification includes a Drug Utilization Review (DUR).

6. The computer-implemented method of claim 3, wherein the verification comprises processing an input that includes the clinical data by a machine learning model that is configured to verify the request.

7. The computer-implemented method of claim 5, further comprising:
adjusting, by the cloud-based medication fulfillment system, the ordered medication based on performing the DUR.

8. The computer-implemented method of claim 1, where processing the representation comprises sending the representation from the cloud-based medication fulfillment system to the automated device via an API that is established by the by the cloud-based medication fulfillment system.

9. The computer-implemented method of claim 1, where processing the representation comprises:
generating, by the cloud-based medication fulfillment system, a code that is associated with preparing the medications of the particular type; and
providing, by the cloud-based medication fulfillment system to the automated device, the code.

10. The computer-implemented method of claim 1, wherein processing the one or more enhanced medication fulfillment steps, by the cloud-based medication fulfillment system, includes, after preparing the medication by the automated compounding device and before the order is completed, decrementing an inventory of ingredients of the medication and incrementing an inventory of the medication.

11. The computer-implemented method of claim 1 wherein processing the request according to one or more legacy medication fulfillment requests includes providing, to a third-party authorization system that is not included in the cloud-based medication fulfillment system, an indication that the order for the medication of the particular type has been fulfilled.

12. The computer-implemented method of claim 1, wherein processing the request according to one or more legacy medication fulfillment requests includes providing, to a third-party infusion-delivery scheduling system that is not included in the cloud-based medication fulfillment system, an indication that the order for the medication of the particular type has been fulfilled.

13. The computer-implemented method of claim 1 further comprising:
obtaining, by the cloud-based medication fulfillment system, from a medication-administration device and though an Application Programming Interface provided by the cloud-based medication fulfillment system, an indication that the medication has been administered; and
providing, by the cloud-based medication fulfillment system and to accounts-receivable system, a charge associated with preparing the medication.

14. The computer-implemented method of claim 3, further comprising canonicalizing the clinical data.

15. A system comprising one or more computers and one or more storage devices storing instructions that when executed by the one or more computers cause the one or more computers to perform operations comprising:
receiving, by a cloud-based medication fulfillment system, a request from a customer to fulfill an order for a medication of a particular type;
when the customer is authorized to process requests via an enhanced medication workflow that includes one or more legacy medication fulfillment steps for fulfilling medications that are not of the particular type as well as one or more enhanced medication fulfillment steps for fulfilling medications that are of the particular type:
processing, by the cloud-based medication fulfillment system, the request according to the one or more legacy medication fulfillment steps, and
processing, by the cloud-based medication fulfillment system, the request according to the one or more enhanced medication fulfillment steps, including processing a representation of the request by an automated device that is associated with preparing medications of the particular type; and
storing, by the cloud-based medication fulfillment system, data indicating that the order for the medication of the particular type has been fulfilled.

16. The system of claim 15, wherein the automated device is an automated compounding device.

17. The system of claim 15, wherein processing the request according to the one or more enhanced medication fulfillment steps comprises:
obtaining, by the cloud-based medication fulfillment system, within a threshold period of time after receiving the request, clinical data;
performing, by the cloud-based medication fulfillment system, and using the clinical data, a verification; and
in response the verification, providing, by the cloud-based medication fulfillment system, a representation of the request to the automated device.

18. The system of claim 17, wherein the verification comprises processing an input that includes the clinical data by a machine learning model that is configured to verify the request.

19. The system of claim 17, the operations further comprising canonicalizing the clinical data.

20. One or more non-transitory computer-readable storage media storing instructions that when executed by one or more computers cause the one or more computers to perform operations comprising:
receiving, by a cloud-based medication fulfillment system, a request from a customer to fulfill an order for a medication of a particular type;
when the customer is authorized to process requests via an enhanced medication workflow that includes one or more legacy medication fulfillment steps for fulfilling medications that are not of the particular type as well as one or more enhanced medication fulfillment steps for fulfilling medications that are of the particular type:
processing, by the cloud-based medication fulfillment system, the request according to the one or more legacy medication fulfillment steps, and
processing, by the cloud-based medication fulfillment system, the request according to the one or more enhanced medication fulfillment steps, including processing a representation of the request by an automated device that is associated with preparing medications of the particular type; and
storing, by the cloud-based medication fulfillment system, data indicating that the order for the medication of the particular type has been fulfilled.
